(19) United States
(12) Patent Application Publication
(10) Pub. No.: US 2004/0156830 A1
(43) Pub. Date: Aug. 12, 2004

(54) Vector and pharmaceutical composition containing cell death inducing gene

(76) Inventor: Heiichiro Udono, Nagasaki-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/769,856

(22) Filed: Feb. 3, 2004

(30) Foreign Application Priority Data
Feb. 3, 2003 (JP) 2003-025909

(51) Int. Cl. A61K 48/00; C12N 15/86

(52) U.S. Cl. 424/93.2; 514/44; 435/456

(57) ABSTRACT

The present invention provides a cell-death-inducing vector containing a gene encoding PA28. The present invention provides a cell-death-inducing agent characterized by comprising the cell-death-inducing vector. The present invention further provides an antitumor pharmaceutical composition characterized by comprising the cell-death-inducing vector.

### CROSS-REFERENCE TO RELATED APPLICATIONS

This application is based upon and claims the benefit of priority from the prior Japanese Patent Application No. 2003-025909, filed Feb. 3, 2003, the entire contents of which are incorporated herein by reference.

### BACKGROUND OF THE INVENTION

1. Field of the Invention

The present invention relates to a vector containing a gene encoding protein activator 28 (PA28). More particularly, the present invention relates to a pharmaceutical composition containing a cell-death-inducing gene, PA28α.

2. Description of the Related Art PA28 is a 20S-proteasome-activating molecule. PA28 is present in the cytoplasm as a hetero-oligomer composed of two subunits of PA28α and PA28β mutually associated. PA28 is also known to associate with both ends of 20S proteasome to form a football-form proteasome. The football-form proteasome is still inactive in protein-degradation activity but augmented in the peptidase activity. This fact suggests that the football-form proteasome is clearly involved in rapid and optimal treatment for endogenous antigens. In particular, it is said that the football-form proteasome may play an important role in peptide production mediated by an MHC class-I molecule. In short, it has been considered that the PA28 molecule may be a regulatory factor for accelerating a degradation activity of a peptide but not a protein.

However, it has been recently found that a proteasome complex (called a hybrid proteasome), which is composed of 20S proteasome and PA28 and PA 700 bound at both ends thereof, is present in the cytoplasm. PA 700 recognize and binds to a ubiquitinated protein. Since this finding, the possibility that the PA28 molecule may be directly involved in protein-degradation itself has been suggested.

On the other hand, as gene therapy for a tumor, attempts have been so far made to introduce genes, such as interferon γ, IL2, IL12, and B7 genes, into cells. In this way, cancers are treated by introducing various cytokine-genes. For example, interferon γ directly suppresses cancer-cell growth and exhibits cytotoxicity to cancer cells, in vitro. These cytokines have been used as cancer vaccines (Fearon et al., “Interleukin-2 production by tumor cells bypasses T helper function in the generation of an antitumor response”, Cell (USA), 1990, Vol. 60, p 397-403; Meijer et al., “Adoptive cellular therapy with tumor vaccine draining lymph node lymphocytes after vaccination with HLA-B7/beta 2-microglobulin gene-modified autologous tumor cells”, J. Immunother., 2002, Vol. 25, p.359-72).

As another attempt, gene therapy for tumor cells is performed by introducing a cytokine gene into lymphocytes such as lymphokine-activated killer cells (LAK), cytotoxic T cells (CTL), and tumor invasive lymphocytes (TIL), allowing a cytokine to secrete from the cytokine-gene introduced cells, activating the lymphocytes by the cytokine thus secreted (called autocrine or paracrine), thereby enhancing passive immunological competence.

As another attempt of gene therapy, an IFN gene or TNF gene having an antitumor activity is directly introduced into the tumor to locally enhance an antitumor activity (Nishihara et al., “Augmentation of tumor targeting in a line of glioma-specific mouse cytotoxic T-lymphocytes by retroviral expression of mouse gamma-interferon complementary DNA”, Cancer Res., (USA), 1988, Vol. 48, p.4730).

However, the introduction of any gene activating an immune effecter mechanism is directed to activating T-cells capable of recognizing cancer cells and is not directed to altering the nature of the cancer cells themselves. Conversely, the introduction of the gene which directly suppresses the proliferation of cancer cells; however would not change the antitumor effect mediated by the immune effecter mechanism.

The present invention has been made with a view to solving the aforementioned problems and is intended to provide a pharmaceutical composition for cancer gene-therapy capable of exhibiting an antitumor activity and activating T-cell immune system.

### BRIEF SUMMARY OF THE INVENTION

The inventors have conducted intensive studies with a view to solving the aforementioned problems and obtained the following findings.

More specifically, We found that the expression levels of cell-cycle mediating molecules, such as cdc2, kip1, cdk4, and survivin, are significantly low in the PA28-gene introduced cells, although the mRNA level is constant; on the other hand, the expression levels of the cell-cycle mediating molecules are high in the PA28 dominant negative gene introduced cells. In addition, in the PA28 dominant negative gene introduced cells, ubiquitination of the above proteins was observed. These facts clearly show that PA28 can accelerate the degradation of these protein, and that the introduction of PA28 gene causes cell-cycle abnormality, leading to apoptosis. To be surprised, when PA 28 is expressed excessively in a tumor, apoptosis is induced in vitro and revealed by mouse tumor transplantation experiment that a tumor is retrograded and rejected.

As described above, PA28 is involved in degradation of many protein molecules, consequently causing a phenomenon of apoptosis in cancel cells. This is a novel mechanism of inducing apoptosis.

When the PA28 gene is introduced in to cancer cells, it immediately causes abnormality in the cell cycle, more specifically, inhibits the cell division in the M phase, followed by causing acceleration of apoptosis. On the other hand, in the PA28 gene introduced cancer cells, MHC class I molecules are significantly expressed, increasing the production of cancer-antigen peptides, and then strong recognition of the antigens by cytotoxic T cells can be made. Therefore, when a PA28 gene is introduced into cancer cells, it is possible to induce the death of the cancer cells themselves and simultaneously activate the T-cell immune system.

Based on the fact described above that when a PA28 gene is introduced in cancer cells using a virus vector containing the PA28 gene and allowed to over-express PA28 gene, delay of cancer cell-cycle and induction of apoptosis occurred, the cancer cells introduced PA28 gene were successfully retrograded and rejected in the early stage. In this way, the present invention was accomplished.

More specifically, the present invention provides a cell-death inducing vector containing a gene encoding PA28.

By introducing the cell-death-inducing vector or cell-death-inducing agent of the present invention into cells (particularly, cancer cells), it is possible to induce abnormality in the cell cycle, causing apoptosis.

Furthermore, by introducing an antitumor pharmaceutical composition of the present invention into tumor cells of a cancer patient, it is possible to induce an antitumor activity, that is, retrogradation and rejection of the tumor cells. In other words, apoptosis is induced in the cells to which the antitumor pharmaceutical composition is introduced, and simultaneously, PA28 activates proteasome, increasing the expression of MHC class I molecules, followed by increasing the production of cancer-antigen peptides. As a result, strong recognition of the antigen by cytotoxic T cells can be made. In short, not only apoptosis of cancer cells themselves can be induced but also the T-cell immune response can be activated.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the preferred embodiments given below, serve to explain the principles of the invention.

FIG. 1 shows the results of a cell cycle analysis of PA28α gene introduced cells;

FIG. 2 shows the apoptosis of PA28α gene introduced cells;

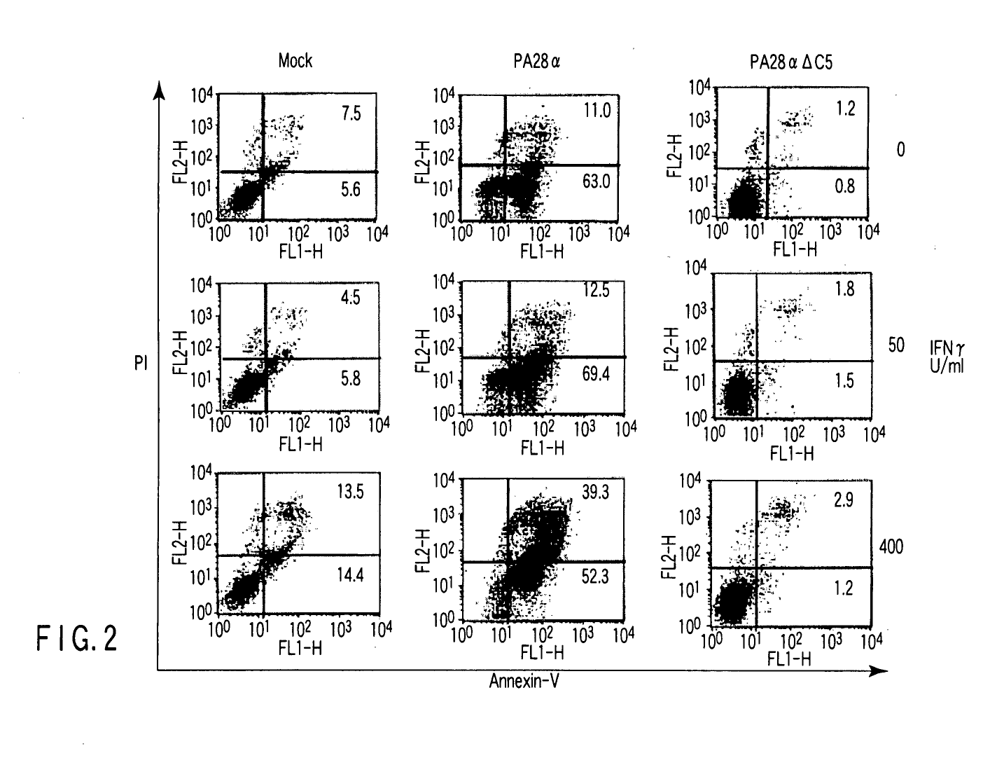

FIG. 3 shows the DNA fragmentation in PA28α gene introduced cells; and

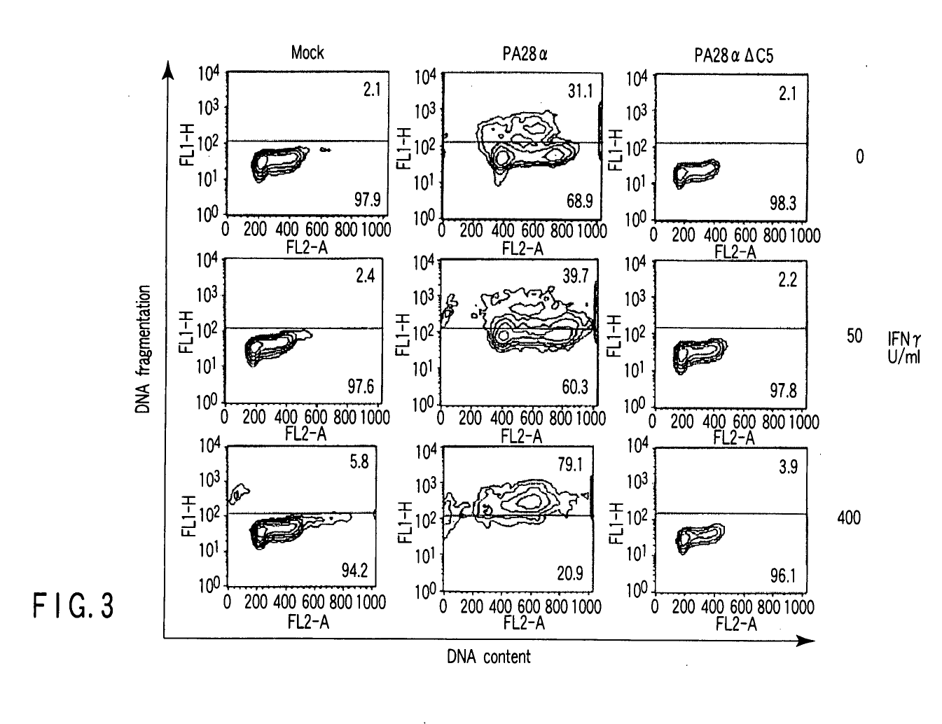

FIG. 4 shows the effect of in-vivo introduction of a PA28α gene.

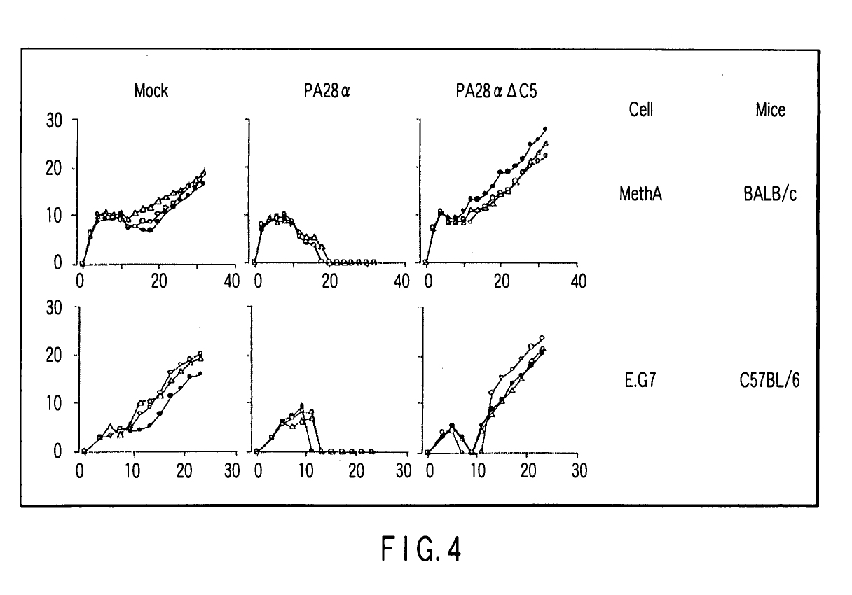

### DETAILED DESCRIPTION OF THE INVENTION

The present invention will be explained in detail below.

A cell-death-inducing vector of the present invention is comprising a PA28 gene. The cell-death-inducing agent and antitumor pharmaceutical composition of the present invention is comprising a vector containing a PA28 gene (the cell-death-inducing vector) as an active ingredient. The cell-death-inducing vector used herein is a vector inducing apoptosis, in other words, a vector inducing apoptosis of a cell when the vector is introduced in the cell. Therefore, when the cell-death-inducing vector, cell-death-inducing agent, or antitumor pharmaceutical composition of the present invention is introduced into a target cell, a delay or the cell cycle arrest of the target cell takes place, causing apoptosis.

The apoptosis is characterized in that a chromosomal DNA of a cell is fragmented into nucleosomes. The apoptosis is determined by detecting DNA fragmentation by a known method such as Annexin-V staining analysis, TUNEL (terminal deoxynucleotidyl transferase-mediated dUTP nick-end-labeling) analysis, or nuclear staining.

The PA28 gene used in the present invention may be a PA28α gene, a PA28β gene, or both; however, the PA28α gene is preferably used. As used herein, the PA28 gene (preferably PA28α) may derived from, but not limited, mammalians such as humans, mice, rats, and rabbits.

The PA28 gene of the present invention may be analogues of PA28 gene. As long as analogues of PA28 gene has substantially the same biologically activity as that of a PA28 gene, it may be used. To describe more specifically, the PA28 gene of the present invention includes genes encoding an amino acid sequence, one or several amino acids of which may be deleted from, replaced for or added to. Therefore, genes encoding an amino acid sequence having a homology of 90% or more, preferably 95% or more, more preferably 97% or more to that of human PA28 are included in the scope of the present invention. Furthermore, the PA28 gene analogues of the present invention may include DNA capable of hybridizing with the gene encoding human PA28 under stringent conditions. In these analogous genes, a mutation may be introduced into the DNA encoding PA28 (preferably PA28α) by a known genetic-engineering technique such as a site-directed mutation/PCR method (e.g., a conventional recombination technique as described in Sambrook, Fritsch and Maniatis, “Molecular Cloning, A laboratory Manual, 2nd Ed. (Cold Spring Harbor Press, Cold Spring Harbor, N.Y., 1989). As the stringent conditions mentioned above, the hybridization conditions described in the above documents may be used. To describe more specifically, hybridization is performed under the conditions: a formamide concentration of 45% (v/v), a salt concentration of 5×SSPE, and a temperature of 42° C.; and washing is performed under the conditions: a salt concentration of 2×SSPE, and a temperature of 42° C.

A gene (cDNA) encoding PA28 used in the present invention is obtained by the following method.

The PA28 molecule is generally known to be more or less expressed in normal cells and also in cancer cells. Therefore, total mRNA is extracted from these cells and then purified. More specifically, the cells are homogenized in a phenol or phenol-chloroform solution containing guanidine isothiocyanate. The resultant solution is centrifuged to separate into an aqueous phase and an organic phase, and then isopropanol is added to isopropanol the aqueous phase containing total mRNA, and precipitating and recovering total mRNA. Alternatively, total mRNA is recovered by sucrose or cesium chloride density gradient centrifugation. A cDNA is then synthesized by a reverse transcription reaction mediated by a reverse transcriptase using the total mRNA (that is, poly (A) RNA) as a template and an oligo (dT) as a primer,

The cDNA is digested, for example, with restriction enzymes to form restriction sites suitable for linkage with a phage and a plasmid vector and then linked with the phage or the plasmid vector having the same restriction sites. The vector thus obtained is introduced into Escherichia coli (E. coli) to transform the E. coli. In this manner, a cDNA library is prepared. Alternatively, since a wide variety of cDNA libraries are constructed from various types of cells and are commercially available, such a cDNA library may be used.

A cDNA library includes DNA fragments having difference information other than the fragment of a target DNA. Therefore, the DNA encoding PA28 (preferably PA28α) must be screened. Since the sequence of the PA28 gene has been already known, a primer is constructed so as to amplify a sequence containing a full-length PA28 based on the sequence information and subsequently a PCR is performed using the primer and the cDNA library, thereby obtaining only the DNA encoding PA28α. For example, to amplify the human PA28α cDNA, ten nucleotides complementary to the sequences of the positions 1 to 20 and positions 735 to 750 of human PA28α cDNA are prepared as primers. Subsequently, PCR is performed using the primers and the library obtained above as a template, thereby specifically amplifying PA28αDNA.

PCR is performed, for example, by repeating a cycle of denaturation at 94° C. for one minute, annealing at 58° C. for one minutes, and extension at 72° C. for one minute, at least 20 times, preferably, 30 times. After cloning and amplifying a target PA28 DNA, the cloned and amplified PA28 DNA is recovered and purified by electrophoresis. Thereafter, the purified DNA is inserted into an appropriate expression vector. In this way, the cell-death-inducing vector of the present invention can be prepared.

As the expression vector to be used as the cell-death-inducing vector of the present invention, any vector may be used as long as it can express a PA28 gene. For example, either a viral vector or a nonviral vector may be used; however, a viral vector is preferably used.

The viral vector, may be a recombinant adenoviral vector and retrovirus vector. More specifically, the viral vector may include DNA viruses such as non-toxicated retrovirus, adenovirus, adeno-associated virus, herpes virus, vaccinia virus, poxvirus, poliovirus, simbis virus, Sendai virus, SV40, and human immunodeficiency virus; and RNA viruses. Of the viral vectors, adeno virus is known to have a considerably higher transfection efficiency than other viral vectors. In view of the transfection efficiency, an adenovirus vector is preferably used.

On the other hand, examples of the non-viral vector include vectors for mammalian cells.

The cell-death-inducing vector of the present invention can be constructed by inserting the amplified PA28 gene (preferably PA28α) into an appropriate expression vector. Any means may be used to insert a PA28 gene into a vector. For example, the vector of the present invention may be constructed by inserting a PA28 gene into an expression vector by use of a conventional recombination technique described in Sambrook, Fritsch and Maniatis, “Molecular Cloning, A laboratory Manual, 2nd Ed., (Cold Spring Harbor Press, Cold Spring Harbor, N.Y., 1989), or other homologous recombination techniques may be used. If the expression vector has a multi-cloning site, the PA28 gene may be inserted in the multi-cloning site.

Examples of the cell-death-inducing vector of the present invention may include a replication origin, a selective marker, and a promoter, and if necessary, an enhancer, a transcription termination site (terminator), a ribosome binding site, and a polyadenylation signal.

The selective marker is a gene for providing to a host, a phenotype for screening a transformed cell. For example, a neomycin resistant gene, thymidine kinase gene and dihydrofolic acid reductase gene, may be used as a vector for mammalian cells.

As such a vector, a commercially available vector may be used. Examples of the vector includes viral vectors such as a pMSCV retrovirus vector (Clontech) and non-viral vectors for mammalian, such as pXT1, pSG5 (Stratagene), pSVK3, pBPV, pMSG, pSVL, and SV40 (Pharmacia).

The cell-death-inducing agent and antitumor pharmaceutical composition of the present invention may contain a vector alone and together with other ingredients. As the other ingredients to be used in combination with the vector, other vector (e.g., a vector containing other genes) and a carrier may be used. Furthermore, various pharmaceutically acceptable carriers, which vary depending upon an administration route, may be included as an ingredient.

In the cell-death-inducing agent and antitumor pharmaceutical composition of the present invention, a vector containing a PA28 gene may be mixed with an appropriate carrier, an excipient, and other chemical agent suitable for administering the vector to the cells or a patient to obtain a preparation. By administering the preparation, the expression of the PA28 gene can be enhanced. For example, the injection preparation may be prepared by dissolving a PA28-gene-containing vector in an appropriate solvent, for example, a buffer solution such as phosphate-buffered solution (PBS), physiological saline, or sterilized water. Alternatively, when the vector containing a PA28 gene is administered in the form of a liposome or the like, the vector of the present invention may be prepared into an appropriate liposome made of a cationic lipid such as LIPOFECTIN (trade name) manufactured by Life Technologies Inc., Bethesda, Md., and administered. In particular, if a known drug delivery technique such as a liposome coupled with an antibody is used, the vector (or pharmaceutical composition) of the present invention can be delivered specifically to the target tissue and cells.

It is well known that a viral vector is taken up into a cell in vivo, integrated into the cell, and expresses viral DNA containing insert DNA in the cell. Thus the desired DNA may be introduced into a cell by infecting the cell with a viral vector.

The cell-death-inducing agent and antitumor pharmaceutical composition of the present invention may be introduced directly into a patient by a known method such as in-vivo or ex-vivo method. In the in-vivo method, the agent or pharmaceutical composition of the present invention is introduced directly into a patient body. In the ex-vivo method, target cells are taken out from a patient and the agent or pharmaceutical composition of the present invention is introduced in the cells outside the body and then the cells are returned into the body. When the agent of the present invention is administered by the in-vivo method, it may be administered via an appropriate administration route, which varies depending upon an apoptosis target such as cells, tissues or organs. More specifically, the agent or pharmaceutical composition of the present invention may be administered to intravenously, intraarterially, subcutaneously, intradermally, and intramuscularly and the like, or directly injected into the lesion locally. The agent or pharmaceutical composition of the present invention may be administered in various preparation forms suitable for the administration route to be used.

As a method of introducing a gene into a non-viral vector cell, a phosphorus-calcium coprecipitation method, and a direct DNA injection method using a micro-glass tube, may be used. As a method of introducing a gene into tissues, any known methods may be used. The methods include a gene-introduction method using a liposome, a gene-introduction method mediated by a receptor, a method of shooting a gene (DNA molecule) together with a carrier (metal particle) into a cell by means of a particle gun, and a gene-introduction method mediated by a positively charged polymer. Any recombinant expression vector may be introduced into a cell by such a known method.

The vector of the present invention or a cell-death-inducing agent and antitumor pharmaceutical composition containing the vector is preferably administered directly to a tumor. The present invention makes it possible to induce apoptosis of various cells and tissues, thereby suppressing tumor growth or treating tumors.

### EXAMPLES

### (Material and Method)

1. Establishment of PA28α Gene-expressing Tumor Cell

1.1 Preparation of PA28α Gene

cDNA encoding PA28α was prepared as follows:

Mouse spleen cells were homogenized in a phenol-chloroform solution containing guanidine isothiocyanate. The resultant solution was separated into an aqueous phase and an organic phase by high-speed centrifugation. Isopropanol was added to the aqueous phase to precipitate total mRNA. In this manner, the total mRNA was recovered. Using the total mRNA as a template and an oligo dT as a primer, cDNA was synthesized through a reverse transcription reaction mediated by a reverse transcriptase. In this way, a DNA library was obtained. Using the sequences complementary to the base sequences of the positions 1 to 20 and positions 735 to 750 as primers and the cDNA library constructed above as a template, cDNA encoding PA28α was amplified. A PCR was performed by repeating a cycle of denaturation at 94° C. for one minute, annealing at 58° C. for one minute, and extension at 70° C. for one minute, at least 20 times, preferably, 30 times. The amplified cDNA was purified by a phenol-chloroform-isoamyl alcohol solution, precipitated with ethanol and recovered.

1.2 Integration of PA28α Gene into Retrovirus Vector

The cDNA encoding PA28α prepared above was cloned into 5′ HpaI and 3′EcoRI sites present in the multi-cloning site of a pMSCV retrovirus vector (available from Clonteck). pT67 retroviral package cells were transfected with 10 μl of the obtained vector using DOTAP ribosomal transfection reagent (Behringer Mannheim Co., Ltd). The obtained cells were incubated continuously for 2 weeks in a medium supplemented with 2 μg/ml puromycin, thereby screening virus-producing cells. More specifically, the cells were incubated in a 6-well plate. When cells proliferate and occupy about 80% of the well surface, the medium is replaced with 4 ml of a flesh medium containing no puromycin, thereby removing puromycin. The culture was incubated for a further 48 hours and the supernatant containing retrovirus (1×10⁷ CFU/ml) was recovered.

1.3 Introduction of PA28α Gene into Cancer Cells by Retrovirus Vector

To 2 ml of the supernatant containing a retrovirus obtained above, polybren was added to a concentration of 5 μg/ml. The obtained solution was added to a pellet of 1×10⁶ cancer-cells, stirred well and spread over a 6-well plate. After 24 hours, 2 ml of a fresh culture solution was added to the wells. After 48 hours, 2 ml of the culture solution was gently removed and, instead, 2 ml of a fresh culture solution containing 2.0 μg/ml of puromycin was added thereto. The incubation is continued while the fresh culture solution containing 2.0 μg/ml of puromycin was appropriately added to the culture in order to prevent excessive proliferation. About 2 weeks after puromycin was initially added, cancer cells stably expressing PA28α were obtained.

2. In-vitro Analysis for Cancer Cells Harboring Gene

2.1 Cell Cycle (Example 1)

1×10⁶ cells are centrifuged and the supernatant is removed.

2 ml of 70% ethanol at −20° C. is poured into the cells with the cells are stirred by a vortex. After the resultant mixture is allowed to stand still at −20° C. for 12 hours, it is gently stirred by a vortex and centrifuged. Ethanol is removed and the sedimentary cells are loosen by tapping.

To the obtained solution, 100 μl of PBS (containing 0.2 M Na₂HPO₄ and 4 mM citric acid) is added, mixed, and allowed to stand at room temperature for about 30 minutes.

After the solution is centrifuged, the supernatant is completely removed. The sedimentary cells are loosen by tapping. 1 ml of a PI-RNase solution (containing 10 μg/ml propidium iodide and 10 mg/ml RNase) is added to the cells and stirred gently by a vortex.

After stand at room temperature for 20 minutes, the resultant solution is subjected to analysis by FACScan (Beckton Dickinson, San Joes, Calif. USA). The DNA amount is plotted on the horizontal axis (linear scale) and the number of cells is plotted on the vertical axis. In this way, a histogram is formed.

2.2 Detection of Apoptosis Cells by Annexin V and Propidium Iodide (PI) Staining (Example 2)

1 to 2×10⁶ cells including apoptosis cells were suspended in PBS (−) and centrifuged. After the supernatant is removed, the sedimentary cells are loosen by tapping.

A binding buffer (containing 10 mM HEPES, 150 mM NaCl, 5 mM KCl, 1 mM MgCl₂.6H₂O, and 1.8M CaCl₂.6H₂O) is added to the cells to a concentration of 5×10⁵ cells/ml.

To 490 μl of the cell suspension solution, 5 μl of Annexin V-FITC and 5 μl of a PI solution (containing 250 μl/ml of a binding buffer) are added, gently mixed and placed on ice for 10 minutes, thereby staining the cells. Immediately after the staining, cells are subjected to FACScan analysis.

After the FACScan analysis, a value of FL1 (FITC-Annexin V) is plotted on the horizontal axis (log scale) and FL2 (PI), on the vertical axis.

Cells exhibiting Annexin V stainability (−) and PI stainability (−) are in the early apoptosic stage; and

Cells exhibiting Annexin V stainability (+) and PI stainability (+) are in the late apoptosic stage or necrosis stage

2.3 TUNEL (TdT Assay) (Example 3)

After 1 to 2×10⁶ cells are centrifuged and the supernatant is removed, sedimentary cells are loosened by tapping. To the cells, 200 μl of 1% formaldehyde-PBS at 4° C. is added, mixed by pipetting, and stand on ice for 15 minutes.

After centrifugation, the supernatant is completely removed, 2 ml of 70% ethanol at −20° C. is poured into the sedimentation while the sedimentary cells are stirred by a vortex. After stand for 12 hours, the resultant solution is centrifuged to remove ethanol. Thereafter, a cold PBS is added to the sediment and washed twice by centrifugation.

The supernatant is completely removed and 100 μl of an equilibrium buffer (0.2M potassium cacodylate, 2.5 mM CoCl₂, 0.1 mM DTT, and 0.25 mg/ml BSA) is added to the sediment without tapping, and mixed by pipetting.

After centrifugation, the supernatant is completely removed. To the sedimentary cells, 50 ml of a TdT reaction solution (containing 5 μM of FITC-dUTP and 5 units of TdT) is added without tapping and mixed by pipetting.

The reaction mixture is allowed to react in a constant-temperature bath at 37° C. for one hour in a dark while stirring by pipetting every 15 minutes. Thereafter, the mixture is centrifugally washed twice with a cold PBS.

The supernatant is sufficiently removed and the sedimentation is loosened by tapping. 1 ml of a PI-RNase solution is added to the sedimentary cells and gently stirred by a vortex.

The mixture is allowed to stand still for 20 minutes in a dark and subjected to FACScan analysis. The DNA amount (PI) is dot-plotted on the horizontal axis (linear scale) and the frequency (FITC) of DNA cleavage, on the vertical axis.

3. In-vivo Tumor Transplantation Experiment (Example 4)

PA28α gene-expressing tumor cells, namely, MethA PA28α and E.G7 PA28α; PA28α ΔC5 (PA28α dominant-negative)-expressing tumor cells, namely, MethA PA28α (ΔC5) and E.G7 PA28α (ΔC5); and control cells, MethA mock and E. G7 mock, to which a vector is only introduced, were prepared.

The living cells of MethA mock, MethA PA28α, and MethA PA28α(ΔC5) (1×10⁷ each) were subcutaneously injected (inoculated) to BALB/c mice, and the living cells of E.G7 mock, E.G7 PA28α, and E.G7 PA28α (ΔC5) (1×10⁶ each), to the C57BL/6 mice.

After the injection (inoculation), the long and short diameters of tumors were measured twice to three times per week and averaged. In this way, the tumor-cell proliferation pattern with the passage of time was obtained.

### RESULTS

### EXAMPLE 1

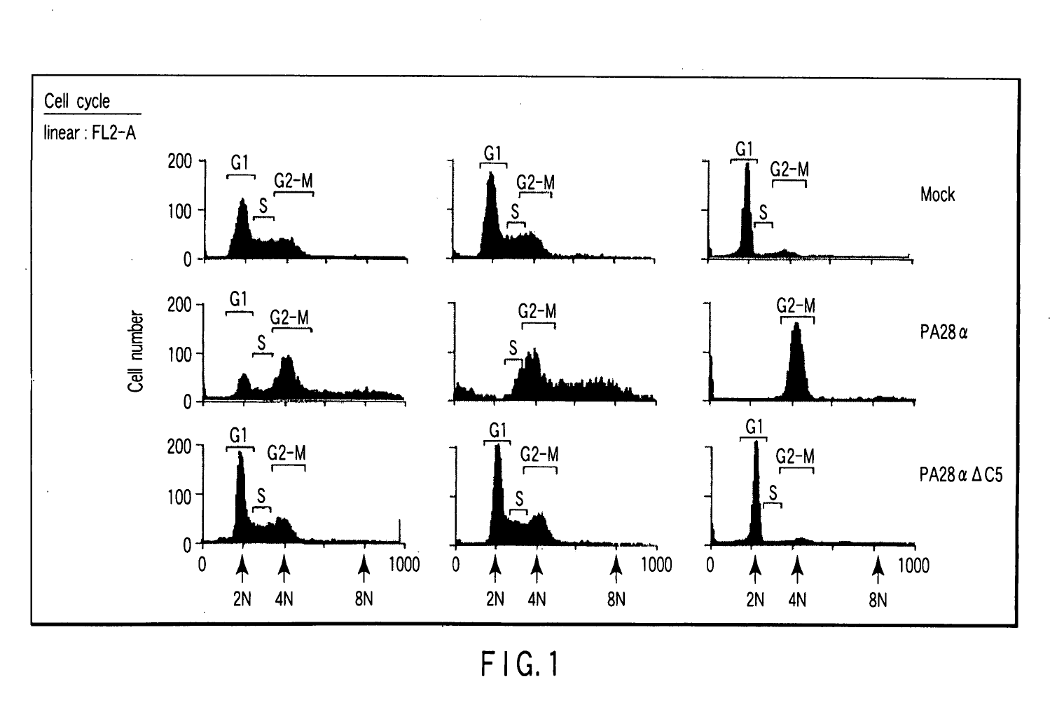

Using a retrovirus vector, PA28α and PA28α ΔC5 (dominant-negative) genes were introduced into mouse tumor cells, E.G7 and MethA, B16. After a drug-resistant stable strain was established, the cell cycle of the strain was analyzed by PI staining. In the PA28α-expressing tumor cells, a delay of the cell-cycle was observed in the G2/M phase. The delay is generally found based on the emergent of 8 polyploid. In the PA28α ΔC5-expressing tumor cells, the G1 phase enhancement was found in every cell (see FIG. 1).

### EXAMPLE 2

Using a retrovirus vector, PA28α and PA28α ΔC5 (dominant-negative) genes were introduced into mouse tumor cells, E.G7. After a drug-resistant stable strain was established, apoptosis analysis was performed by PI staining and Annexin-V staining. It was confirmed that a population of Annexin-V positive cells appeared in the PA28α-expressing tumor cells. The number of the Annexin-V positive cells increases in a concentration-dependent manner in the presence of IFN-γ. PI positive cells come to appear with an increase of the concentration of IFN-γ. In the PA28α ΔC5-expressing tumor cells, it was confirmed that a population of Annexin-V positive cells did not appear even in the presence of IFN-γ.

### EXAMPLE 3

Using a retrovirus vector, PA28α and PA28α ΔC5 (dominant-negative) genes were introduced into mouse tumor cells, E.G7. After a drug-resistant stable strain was, established, the amount of DNA (cell cycle) was detected by PI staining. Furthermore, DNA fragmentation were detected by the TUNEL method. In the PA28α-expressing tumor cells, it was confirmed that undivided cells appeared in the M phase. In addition, DNA fragmentation was observed over the entire cell-cycle. The frequency of DNA fragmentation increased in a concentration-dependent manner in the presence of IFN-γ. On the other hand, in the PA28α ΔC5-expressing tumor cells, it was confirmed that no DNA fragmentation was caused even in the presence of IFN-γ.

### EXAMPLE 4

Using a retrovirus vector, PA28α and PA28α ΔC5 (dominant-negative) genes were introduced into mouse tumor cells, E.G7 and MethA. After a drug-resistant stable strain was obtained, the stable strain was transplanted into the same strain of mice (one group consists of three mice) and tumor proliferation was observed. In the PA28α gene introduced tumor, retrogression and rejection of the tumor were observed, whereas, in the tumor harboring PA28α ΔC5 (dominant negative), the tumor diameter gradually increased after the transplantation.

### DISCUSSION

It was demonstrated that introduction of a PA28α gene causes apoptosis of cancer cells. Furthermore, it was demonstrated in vivo that the introduction of a PA28α gene induces apoptosis of cancer cells and causes retrogression of a tumor and rejection of the tumor-cell growth.

In cancer patients, since a tumor has various immune-avoidance mechanisms, the tumor is free from the attack from the immune system. In addition, a tumor acquires the resistance against apoptosis. In these circumstances, if a PA28 gene is introduced into a tumor, effective gene therapy can be developed.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A cell-death-inducing vector containing a gene encoding PA28.

2. A cell-death-inducing agent comprising a vector according to claim 1.

3. Antitumor pharmaceutical composition comprising a vector according to claim 1.

4. The vector according to claim 1, wherein said PA28 is a PA28α.

5. A pharmaceutical composition comprising the cell-death-inducing agent according to claim 2, wherein said PA28 is a PA28α.

6. A pharmaceutical composition comprising the antitumor pharmaceutical composition according to claim 3, wherein said PA28 is a PA28α.

7. The vector according to claim 1, wherein said vector is a viral vector.

8. The vector according to claim 4, wherein said vector is a viral vector.

9. The cell-death-inducing agent according to claim 2, wherein said vector is a viral vector.

10. The pharmaceutical composition according to claim 5, wherein said vector is a viral vector.

11. The antitumor pharmaceutical composition according to claim 3, wherein said vector is a viral vector.

12. The pharmaceutical composition according to claim 6, wherein said vector is a viral vector.
